(19) United States
(12) Patent Application Publication
(10) Pub. No.: US 2022/0096579 A1
(43) Pub. Date: Mar. 31, 2022

(54) Production and Use of Yeast Extract as a Medical Adjuvant

(71) Applicant: Locus IP Company, LLC, Solon, OH (US)

(72) Inventors: Sean FARMER, Ft. Lauderdale, FL (US); Ken ALIBEK, Solon, OH (US)

(21) Appl. No.: 17/423,862

(22) PCT Filed: Jan. 27, 2020
(86) PCT No.: PCT/US2020/015144

§ 371 Date: Jul. 17, 2021

Related U.S. Application Data
(60) Provisional application No. 62/797,599, filed on Jan. 28, 2019.

(51) Int. Cl. A61K 36/064; A61K 31/05; A61K 39/39

(52) U.S. Cl. CPC A61K 36/064; A61K 2039/55588; A61K 39/39; A61K 31/05

(57) ABSTRACT

The present invention relates to compositions and methods for enhancing bioavailability of health-promoting compounds, such as pharmaceuticals and nutritional supplements. The subject invention utilizes an adjuvant composition comprising yeast extract to enhance bioavailability of health-promoting compounds and to reduce the effective dosage that is required.

### CROSS-REFERENCE TO RELATED APPLICATION

This application claims priority to U.S. Provisional Patent Application No. 62/797,599, filed Jan. 28, 2019, which is incorporated herein by reference in its entirety.

### BACKGROUND OF THE INVENTION

In general, bioavailability can refer to the rate and extent to which a substance reaches and enters a desired body system of a living organism, and can be effective therein. Specifically, bioavailability in the context of pharmacology is a measure of the rate and extent to which a drug reaches a site of action. In the realm of nutrition, bioavailability for food and dietary supplements can be defined as the proportion of an administered substance (or ingested substance) capable of being absorbed by the body and which is then available for use or storage in the body. Furthermore, bioavailability can also be the measure by which certain substances from the environment enter a living organism.

The bioavailability of a substance can play an important role in its usefulness for a living organism, and can change based on a variety of factors. For example, the bioavailability of ingested substances can be affected by the solubility of the substance, the rejection of the substance by the epithelium, or the speed at which the substance enters through the layers of the gastrointestinal (GI) tract. Substances with low solubility may not have a sufficient retention time, as they are incapable of penetrating either through the cells or the tight junctions between the cells of the GI tract. Thus, most, if not all of the substance may be released from the body, unabsorbed and unused.

In addition to solubility, rejection of the substance is another factor affecting bioavailability. For example, many substances can be rejected by P-glycoprotein 1, a protein of the cell membrane that pushes foreign substances out of cells. More formally, it is an ATP-dependent efflux pump with broad substrate specificity. This pump is thought to have evolved as a defense mechanism against harmful substances, but can serve as an obstacle in many cases when a foreign, yet desirable, substance is sought to be introduced into the body. It is broadly distributed and expressed in the cells of a variety of organs, including the intestinal epithelium, where it pumps, for example, xenobiotics, back into the intestinal lumen; in liver cells, where it pumps substances into bile ducts; in the cells of the proximal tubule of the kidney, where it pumps substances into the urine-conducting ducts; and in the capillary endothelial cells composing the blood-brain barrier and blood-testis barrier, where it pumps substances back into the capillaries.

Pharmaceuticals, supplements and nutrition are important aspects of leading a healthy life; however, the dosage or amount of certain health-promoting compounds that must be administered to a subject is often far greater than is actually needed to have a desired effect. This is, in part, because evolutionary obstacles hinder the bioavailability of certain compounds and nutrients from reaching a desired site of action, for example, through epithelial cells and through the blood-brain barrier.

Thus, there is a need for compositions and methods that are capable of enhancing the bioavailability of a broad range of pharmaceuticals, supplements, nutrients and other health-promoting compounds.

### BRIEF SUMMARY OF THE INVENTION

The subject invention provides materials and methods for improving the bioavailability of pharmaceuticals, supplements, nutrients and/or other health-promoting compounds. In particular, the subject invention provides method for enhancing bioavailability of health-promoting compounds using microbe-based products. Advantageously, the microbe-based products and methods of the subject invention are non-toxic and cost-effective.

In certain specific embodiments, the subject invention provides approaches to enhancing bioavailability of a health-promoting compound, for example, by suppressing P-glycoproteins and/or modulating other physical barrier mechanisms that would otherwise reduce the penetration of certain substances into, for example, a subject's epithelial cells and/or across the blood-brain barrier (BBB).

In preferred embodiments, methods are provided for enhancing the bioavailability of a health-promoting compound to a subject in need thereof, wherein the method comprises administering an adjuvant composition comprising a yeast extract to the subject, and administering the health-promoting compound to the subject. In certain embodiments, the yeast extract comprises the remaining components of yeast cells and, optionally, their growth by-products, after lysis of the cells and removal of cell walls. In certain embodiments, the yeast extract is produced from Saccharomyces cerevisiae, or baker's yeast, that has been cultivated in a nutrient medium comprising one or more oils, such as, for example, vegetable oil, peanut oil or coconut oil.

Health-promoting compounds comprise any molecule or molecules that are meant to be delivered into blood and/or lymphatic circulation, as well as into tissues and organs, and ultimately reach a site in a subject's body where a positive impact on the subject's health can be effected. Non-limiting examples of health-promoting compounds include pharmaceuticals and/or nutritional supplements categorized as pain-relievers, antihistamines, antivirals, anticancer and/or chemotherapeutic compounds, antibiotics, antimicrobials, antiseizure compounds, anti-inflammatory compounds, vaccines, statins, antidepressants, vitamins, minerals, nutrients, water and many others.

In one embodiment, the health-promoting compound is an orally deliverable health-promoting compound, which, in particular, is any molecule or molecules that is delivered via initial absorption into the gastrointestinal tract or into the mucus membranes of the mouth (e.g., by way of sublingual or buccal administration).

In some embodiments, the adjuvant composition can be mixed with the health-promoting compound. Alternatively, the adjuvant composition can be a separate composition from the target health-promoting compound, wherein the adjuvant composition is intended to be administered to a subject separately, but concurrently with or close in time to, the health-promoting compound.

In one embodiment, the adjuvant composition and/or the health-promoting compound are formulated as an orally-consumable product, such as, for example, a capsule, a pill or a drinkable liquid. Advantageously, the methods can be used to allow for oral administration of health-promoting compounds that might otherwise by degraded by acids or enzymes in the GI tract.

In another embodiment, the adjuvant composition and/or the health-promoting compound are formulated to be administered via injection, suppository, inhalation, or any other mode of administration.

In certain embodiments, the materials and methods of the subject invention can help improve the quality of life for individuals who are either suffering from a particular health condition or who are already healthy (e.g., generally free from illness or injury) but are simply seeking to enhance their state of being. Furthermore, the subject invention can be used to reduce the dosage of certain pharmaceuticals and/or supplements that are required to be considered therapeutically-effective, thus reducing the cost and potential toxicity and/or negative side-effects that might arise from administering them to a subject.

### DETAILED DESCRIPTION

The subject invention provides materials and methods for improving the bioavailability of pharmaceuticals, supplements, nutrients and/or other health-promoting compounds. In particular, the subject invention provides adjuvant compositions comprising microbe-based products for use in enhancing bioavailability of health-promoting compounds. Advantageously, the microbe-based products and methods of the subject invention are non-toxic and cost-effective.

### Selected Definitions

As used herein, the term “adjuvant” in the context of the subject compositions means an auxiliary compound that can aid in, contribute to, and/or enhance the effectiveness of a substance that is administered with the adjuvant. For example, an adjuvant can be taken alongside or included in a prescription drug or a supplement to aid in the effectiveness of the active, primary active ingredient(s), whatever the purpose may be (e.g., treating a disease or enhancing the functioning of an organ or system in the body).

As used herein, the term “subject” refers to an animal, such as a mammal, needing or desiring delivery of the benefits provided by a health-promoting compound. The animal may be for example, pigs, horses, goats, cats, mice, rats, dogs, apes, fish, chimpanzees, orangutans, guinea pigs, hamsters, cows, sheep, birds, e.g., chickens, as well as any other vertebrate or invertebrate animal. The benefits needed or desired can include, but are not limited to, treatment of a health condition, disease or disorder; prevention of a health condition, disease or disorder; hydration or rehydration; nutritional enhancement and/or supplementation for, e.g., athletic performance or weight control; immune health; enhancement of function of an organ, tissue or system in the body; and/or simply pleasure. In certain embodiments, the preferred subject in the context of this invention is a human. In some embodiments, the subject is suffering from a health condition, disease or disorder, while in other embodiments, the subject is in a state of good health (i.e., free from injury or illness), but desires enhanced health and/or functioning of an particular organ, tissue or body system. The subject can be of any age or stage of development, including infant, toddler, adolescent, teenager, adult, and senior.

As used herein, the terms “therapeutically-effective amount,” “therapeutically-effective dose,” “effective amount,” and “effective dose” are used to refer to an amount or dose of a compound or composition that, when administered to a subject, is capable of treating or improving a condition, disease or disorder in a subject, or that is capable of providing enhancement in health or function to an organ, tissue or body system. In other words, when administered to a subject, the amount is “therapeutically effective.” The actual amount will vary depending on a number of factors including, but not limited to, the particular condition, disease or disorder being treated or improved; the severity of the condition; the particular organ, tissue or body system of which enhancement in health or function is desired; the size, age, and health of the patient; and the route of administration.

As used herein, the term “treatment” refers to eradicating, reducing, ameliorating, or reversing a sign or symptom of a health condition, disease or disorder to any extent, and includes, but does not require, a complete cure of the condition, disease or disorder. Treating can be curing, improving, or partially ameliorating a disorder. “Treatment” can also include improving or enhancing a condition or characteristic, for example, bringing the function of a particular system in the body to a heightened state of health or to homeostasis.

As used herein, “preventing” a health condition, disease or disorder refers to avoiding, delaying, forestalling, or minimizing the onset of a particular sign or symptom of the condition, disease or disorder. Prevention can, but is not required to be, absolute or complete, meaning the sign or symptom may still develop at a later time. Prevention can include reducing the severity of the onset of such a condition, disease or disorder, and/or inhibiting the progression of the condition, disease or disorder to a more severe condition or disorder.

As used herein, reference to a “microbe-based composition” means a composition that comprises components that were produced as the result of the growth of microorganisms or other cell cultures. A microbe-based composition may comprise the microbes themselves, or the microbes may be separated from the medium in which they were cultivated, and the composition comprises residual cellular components and/or by-products of microbial growth.

The subject invention further provides “microbe-based products,” which are products that are to be applied in practice to achieve a desired result. The microbe-based product can be simply the microbe-based composition. Alternatively, the microbe-based product may comprise further ingredients that have been added or may be removed of some components. Additional ingredients can include, for example, stabilizers, buffers and/or appropriate carriers (e.g., water or salt solutions). The microbe-based product may comprise mixtures of microbe-based compositions. The microbe-based product may also comprise one or more components of a microbe-based composition that have been processed in some way such as, but not limited to, extraction, filtering, centrifugation, lysing, drying, purification and the like. In certain embodiments, the microbe-based products according to the subject invention comprise “yeast extract,” which, as used herein, comprises the components of a yeast culture (e.g., proteins, amino acids, nucleic acids, carbohydrates, vitamins, minerals, metabolites) after the yeast cells have been lysed and the cell walls removed.

As used herein, an “isolated” or “purified” nucleic acid molecule, polynucleotide, polypeptide, protein or organic compound such as a small molecule (e.g., those described below), is substantially free of other compounds, such as cellular material, with which it is associated in nature. In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state.

A “metabolite” refers to any substance produced by metabolism (e.g., a growth by-product) or a substance necessary for taking part in a particular metabolic process. A metabolite can be an organic compound that is a starting material, an intermediate in, or an end product of, metabolism.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19, or 20 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 1%, 5%, 10%, 25%, 50%, 75%, or 100%.

By “reference” is meant a standard or control condition.

As used herein, a “biofilm” is a complex aggregate of microorganisms, wherein the cells adhere to each other. Biofilms can also adhere to surfaces. The cells in biofilms are physiologically distinct from planktonic cells of the same organism, which are single cells that can float or swim in liquid medium.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention, e.g., the ability to improve the bioavailability of a substance. Use of the term “comprising” contemplates embodiments that “consist” or “consist essentially” of the recited element(s).

Unless specifically stated or obvious from context, as used herein, the term “or” is understood to be inclusive. Unless specifically stated or obvious from context, as used herein, the terms “a,” “an” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Methods of Enhancing Bioavailability of Health-Promoting Compounds

The subject invention provides methods of enhancing the bioavailability of a health-promoting compound in a subject in need thereof, the methods comprising administering a therapeutically-effective amount of a microbe-based adjuvant composition to the subject and administering a therapeutically-effective amount of the health-promoting compound to the subject.

Advantageously, the materials and methods of the subject invention can help improve the quality of life for individuals who are either suffering from a particular health condition or who are already healthy (e.g., generally free from illness or injury) but are simply seeking to enhance their state of being. Additionally, the method can be used to allow for oral administration of health-promoting compounds that might otherwise by degraded by acids or enzymes in the GI tract. Furthermore, the subject invention can be used to reduce the dosage of certain pharmaceuticals and/or supplements that are required to be considered therapeutically-effective, thus reducing the cost and potential toxicity and/or negative side-effects that might arise from administering them to a subject.

In preferred embodiments, the methods of the present invention comprise administering a microbe-based adjuvant composition comprising a yeast extract to the subject, and administering the health-promoting compound to the subject.

In certain embodiments, the yeast extract comprises the remaining components of yeast cells and, optionally the growth by-products of the yeast, after lysis of the cells and removal of cell walls. In certain embodiments, the yeast extract is produced from Saccharomyces cerevisiae, or baker's yeast, using known methods, wherein prior to lysing the cells, the yeast is cultivated in a nutrient medium comprising an oil, such as, for example, vegetable oil, peanut oil or coconut oil.

The microorganisms according to the subject methods may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

The health-promoting compound can be administered simultaneously with the adjuvant composition, for example, as part of a single mixture or formulation. In one embodiment, the adjuvant composition and the health-promoting compound are formulated together into a capsule, nanocapsule or liposome. In one embodiment, additional biological polymers can be included to provide further structure for encapsulation.

Alternatively, the health-promoting compound can be administered as a separate composition from the adjuvant composition. In this alternate embodiment, the health-promoting compound is administered either concurrently with, immediately before or immediately after the adjuvant composition is administered, wherein “immediately before” or “immediately after” means 5 minutes, 4 minutes, 3 minutes, 2 minutes, 1 minute, 30 seconds or less before or after.

Advantageously, in certain embodiments, a target health-promoting compound can exhibit resistance to degradation by digestive juices (e.g., acids and enzymes) when administered into the gastrointestinal (GI) system along with the subject adjuvant composition. Furthermore, in certain embodiments, the subject adjuvant composition can help suppress and/or modulate the activity of, for example, blood plasma proteins, P-glycoproteins, and other barriers and cell junctions that prevent compounds from penetrating into a target site of the body.

The subject invention is useful for enhancing the bioavailability of “health-promoting compounds,” which comprise any molecule or molecules that are meant to be delivered into blood and/or lymphatic circulation, as well as into tissues and organs, and ultimately reach a site in a subject's body where a positive impact on the subject's health, either locally or systemically, can be effected. Health-promoting compounds include, for example, any category of supplement and/or pharmaceutical (including biopharmaceuticals) used for, for example, relieving pain, fever and/or inflammation; reducing the symptoms of allergies or colds; suppressing or treating a virus; treating cancer; treating a microbial infection; suppressing or preventing seizures; lowering or managing cholesterol; managing diabetes; treating depression or anxiety; hydrating or rehydrating; promoting sleep; controlling body weight; enhancing athletic performance; and reducing or enhancing fertility, to name just a few.

The health-promoting compound can be, for example, a pharmaceutical compound, a nutritional supplement, or even simply water. In one embodiment, the subject compositions are formulated as an orally-consumable product, such as, for example, a capsule, a pill or a drinkable liquid. In another embodiment, the subject compositions are formulated to be administered via injection, inhalation, or any other mode of administration.

As used herein, “administering” a composition or product refers to delivering it to a subject such that it contacts a target or site such that the composition or product can have an effect on that target or site. The effect can be due to, for example, the action of a health-promoting compound or adjuvant. Administration can be acute or chronic (e.g., daily, weekly, monthly, etc.) or in combination with other agents. The subject adjuvant composition, whether administered in the same formulation as the target health-promoting compound or within, for example, 5 minutes of the target compound, can be administered by any route of administration provided it is formulated for such a route. In this way, the therapeutic effects attainable by the methods and compositions of the invention can be, for example, systemic, local, tissue-specific, etc., depending of the specific needs of a given application of the invention.

In one embodiment, compositions according to the subject invention can be ingested by a subject in order for the compositions to be contacted with the gastrointestinal tract (e.g., the target site) and have a desired local effect therein or to be absorbed therein for systemic effects. Administration can also be achieved through, for example, injection (e.g., intravenous (IV), intramuscular (IM), intraperitoneal, intrathecal or subcutaneous), transdermal, rectal, urogenital (e.g., vaginal), ocular, aural, nasal, mucosal, inhalation and cutaneous routes.

In one embodiment, the health-promoting compound is a supplement. The supplement can be synthetic, or can be naturally-derived, for example, originating from microbial, fungal, plant or animal sources. The supplement can be a dietary and/or nutritional supplement, for example, providing nutrients such as vitamins (e.g., A (retinoids), B1 (thiamine), B2 (riboflavin), B3 (niacin), B5 (panthothenic acid), B6 (pyridoxine), B9 (folic acid), B12 (cobalamin), C (ascorbic acid), D (calciferol), E (tocopherol), H (biotin), K, and/or derivatives thereof); electrolytes and minerals (e.g., calcium, phosphorous, magnesium, potassium, sodium chloride, iodine, zinc, iron, copper, chromium, fluoride, selenium, manganese, and molybdenum); and fats, carbohydrates and/or proteins (e.g., whey, hemp, soy, collagen, amino acids). The supplement can be a source of energy, alertness, and/or increased physical performance, providing, for example, caffeine, yerba mate, creatine and/or guarana. The supplement can also be a botanical or herbal supplement, for example, turmeric root, cannabidiol (CBD) or ginseng, for holistic health benefits.

In one embodiment, the health-promoting compound is water, wherein the adjuvant composition can be administered as an enhanced hydration or rehydration compound to increase the bioavailability and absorption of water in the GI tract. In some embodiments, the water comprises vitamins and/or electrolytes for further enhanced hydration or rehydration.

In one embodiment, the health-promoting compound is a pharmaceutical or biopharmaceutical. As used herein, the phrase “pharmaceutical” refers to a compound manufactured for use as a medicinal and/or therapeutic drug, whether prescribed by a health care professional or available over the counter. As used herein, the phrase “biopharmaceutical” refers to a biological macromolecule or cellular component, such as a blood product, used as a pharmaceutical. Biopharmaceuticals are typically manufactured in, extracted from, or semi-synthesized from biological sources.

In one embodiment, the pharmaceutical is selected from an antiviral (e.g., acyclovir or valacyclovir), an antibiotic (e.g. erythromycin), a pain-reliever and/or anti-inflammatory compound (e.g., ibuprofen or aspirin).

Additional, and non-limiting examples of pharmaceuticals that can be health-promoting compounds according to the subject invention include, analgesics (e.g., NSAIDs, opioids, acetaminophen, naproxen and local anesthetics); muscle relaxants; digestive aids (e.g., antacids, reflux suppressants, PPIs, laxatives, probiotics, prebiotics, and antidiarrheals); cardiovascular drugs (e.g., beta blockers, calcium channel blockers, diuretics, vasoconstrictors, vasodilators, cardiac glycosides, antiarrhythmics, nitrates); blood pressure/hypertension drugs (e.g., ACE inhibitors, alpha blockers, angiotensin receptor blockers); coagulation drugs (e.g., anticoagulants, heparin, antiplatelet drugs, fibrinolytics, anti-hemophilic factors and haemostatic drugs); statins (e.g., LDL cholesterol inhibitors and hypolipidaemic agents); endocrine aids (e.g., androgens, antiandrogens, estrogens, gonadotropin, corticosteroids, HGH, vasopressin); antidiabetics (e.g., sulfonylureas, biguanides, metformin, thiazolidinediones, insulin); thyroid hormones and antithyroid drugs; urogenital system drugs (e.g., antifungals, alkalinizing agents, quinolones, antibiotics, cholinergics, anticholinergics, fertility medications, hormonal contraceptives); central nervous system drugs (e.g., psychedelics, hypnotics, anesthetics, antipsychotics, eugeroics, antidepressants (including tricyclics, monoamine oxidase inhibitors, lithium salts, and SSRIs), antiemetics, anticonvulsants/antiepileptics, stimulants, amphetamines, dopamine agonists, antihistamines, cannabinoids, 5-HT antagonists); ocular medications (e.g., topical anesthetics, sympathomimetics, parasympatholytics, mydriatics, cycloplegics, mast cell inhibitors); antimicrobials (e.g., antibiotics, antibacterials, antifungals, antiparasitics, antiprotozoals, amoebicides); antivirals (e.g., acyclovir, ribavirin, valacyclovir, famciclovir), antihistamines, anticholinergics, antiseptics, cerumenolytics, bronchodilators, antitussives, mucolytics, decongestants, antimalarials, antitoxins, antivenoms, vaccines, immunoglobulins, immunosuppressants, interferons, monoclonal antibodies, chemotherapeutic drugs and/or any other category of compounds that are capable of treating any health condition, disease or disorder, or of enhancing health in any way.

In one exemplary embodiment, the subject invention comprises an adjuvant composition and a health-promoting compound, wherein the adjuvant composition is a yeast extract of S. cerevisiae and the health-promoting compound is cannabidiol (CBD).

Further components can be added to the compositions as are determined by the skilled artisan such as, for example, buffers, carriers, viscosity modifiers, preservatives, flavorings, dyes and other ingredients specific for an intended use. One skilled in this art will recognize that the above description is illustrative rather than exhaustive. Indeed, many additional formulations techniques and pharmaceutically-acceptable excipients and carrier solutions suitable for particular modes of administration are well-known to those skilled in the art.

### Growth of Microbes for Extract Production

The subject invention provides methods for cultivation of microorganisms for the production of microbial extracts. The microbial culture can be obtained by cultivation processes ranging from small to large scales, including, but not limited to, submerged cultivation/fermentation, solid state fermentation (SSF), and hybrids, modifications and/or combinations thereof.

The growth vessel used for growing microorganisms can be any fermenter or cultivation reactor for industrial use. In one embodiment, the vessel may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can further comprise supplementing the cultivation with one or more carbon sources. Typically, the carbon source would be a carbohydrate, such as glucose, sucrose, lactose, fructose, trehalose, mannose, mannitol, and/or maltose; an organic acid such as acetic acid, fumaric acid, citric acid, propionic acid, malic acid, malonic acid, and/or pyruvic acid; and/or an alcohol such as ethanol, isopropyl, propanol, butanol, pentanol, hexanol, isobutanol, and/or glycerol.

In one embodiment, the method comprises use of one or more sources of fatty acids as a carbon source in place of, for example, glucose or others described above. For example, in one embodiment, the carbon source is a saturated fat or oil, such as soybean oil, rice bran oil, canola oil, olive oil, corn oil, sesame oil, linseed oil, vegetable oil, peanut oil, coconut oil, sunflower oil, olive oil, or any other oil suitable for use in, for example, cooking.

In one embodiment, glucose can be used as a carbon source at the beginning of cultivation in order to increase the cell biomass, but upon exhaustion of the glucose supply in the nutrient medium, a saturated fat or oil serves as a source of carbon.

In one embodiment, the microorganisms can be grown on a solid or semi-solid substrate, such as, for example, corn, wheat, soybean, chickpeas, beans, oatmeal, pasta, rice, and/or flours or meals of any of these or other similar substances.

In one embodiment, growth factors and trace nutrients for microorganisms are included in the medium. Inorganic nutrients, including trace elements such as iron, zinc, copper, manganese, molybdenum and/or cobalt may also be included in the medium. Furthermore, sources of vitamins, essential amino acids, and microelements can be included, for example, in the form of yeast extract, flours or meals, such as corn flour, or in the form of extracts, such as potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, calcium carbonate, sodium chloride and/or sodium carbonate. These inorganic salts may be used independently or in a combination of two or more.

In some embodiments, the method for cultivation may further comprise adding additional acids and/or antimicrobials in the liquid medium before and/or during the cultivation process. Antimicrobial agents or antibiotics are used for protecting the culture against contamination.

Additionally, antifoaming agents may also be added to prevent the formation and/or accumulation of foam.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the liquid medium may be necessary.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of the liquid, and air spargers for supplying bubbles of gas to the liquid for dissolution of oxygen into the liquid.

In one embodiment, the method for cultivation of microorganisms is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

The biomass content of the fermentation medium may be, for example from 5 g/l to 180 g/l or more. In one embodiment, the solids content of the medium is from 10 g/l to 150 g/l.

The method and equipment for cultivation of microorganisms and production of the microbial by-products can be performed in a batch, quasi-continuous, or continuous processes.

Advantageously, the methods of cultivation do not require complicated equipment or high energy consumption. The microorganisms of interest can be cultivated at small or large scale on site and utilized, even being still-mixed with their media.

### Preparation of Microbe-Based Products

The subject invention utilizes yeast extract products prepared from yeasts cultivated in the presence of an oil, such as a saturated oil, as a preferred carbon source over, for example, glucose. The fermentation medium containing the microorganism, its growth by-products, and/or any residual nutrients can be subjected to extraction, concentration, isolation and/or purification using standard methods or techniques described herein and/or in the literature.

The microbes and/or medium (including discrete layers or fractions) resulting from the microbial growth can be removed from the growth vessel and transferred via, for example, piping for processing into yeast extract.

In other embodiments, the composition (microbes, broth, or microbes and broth) can be placed in containers of appropriate size, taking into consideration, for example, the intended use, the contemplated method of application, the size of the fermentation tank, and any mode of transportation from microbe growth facility to the location of use. Thus, the containers into which the microbe-based composition is placed may be, for example, from 1 gallon to 1,000 gallons or more. In other embodiments the containers are 2 gallons, 5 gallons, 25 gallons, or larger.

Upon harvesting the microbe-based composition from the growth vessels, further components can be added as the harvested product is placed into containers and/or piped (or otherwise transported for use). The additives can be, for example, buffers, carriers, other microbe-based compositions produced at the same or different facility, viscosity modifiers, preservatives, pH modifiers, nutrients for microbe growth other ingredients specific for an intended use.

Advantageously, in accordance with the subject invention, the microbe-based product may comprise medium in which the microbes were grown. The product may be, for example, at least, by weight, 1%, 5%, 10%, 25%, 50%, 75%, or 100% broth. The amount of biomass in the product, by weight, may be, for example, anywhere from 0% to 100%, 1% to 99%, 5% to 95%. 10% to 90%, 20% to 80%, 30% to 70%, 40% to 60%, or 50% to 55%.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C.

In certain embodiments, the microbe-based product is a yeast extract produced by: a) cultivating yeast cells in a nutrient medium comprising one or more oils as a carbon source to produce a desired cell density; b) inactivating the yeast cells to produce a hydrolysate; c) centrifuging the hydrolysate to produce a supernatant and a pellet comprising yeast cell walls; and d) removing the pellet. The supernatant after removal of the pellet comprises the yeast extract.

According to the subject invention, a “hydrolysate” of a microorganism comprises disrupted cell walls/membranes of a deactivated microorganism, along with the cell contents released therefrom. The process of deactivating, or hydrolysis, often causes the release of compounds from the cells and cell walls/membranes, such as metabolites, enzymes, proteins, peptides, free amino acids, vitamins, minerals and trace elements. These released compounds are present in the aqueous supernatant yeast extract.

In one embodiment, the hydrolysates are autolysates, wherein a mode of inactivation in d) is chosen such that the mode of inactivation does not inactivate or denature the microorganism's endogenous digestive enzymes, and wherein the endogenous digestive enzymes activate autolysis of the microbial cells.

Inactivating the microorganism can be achieved using, for example, boiling, dry-heat oven, autoclaving, pasteurization, salinization, refrigeration, freezing, high-pressure processing, hyperbaric oxygen therapy, desiccation, lyophilization, radiation, sonication, HEPA (high-efficiency particulate air) filtration, or membrane filtration.

In some embodiments, the yeast extract is produced from the yeast slurry waste of brewing beer (“spent” brewer's yeast). After the beer liquor is removed, for example, by centrifuging the yeast slurry, the slurry can be subjected to hydrolysis and/or autolysis and centrifuged to remove the cell walls.

In some embodiments, the yeast extract is further concentrated. Concentration can be achieved using, for example, a rotary evaporator, wherein the yeast extract is loaded into the rotary evaporator, and the rotary evaporator is operated at a low temperature, e.g., 40 to 80° C., 45 to 75° C., or 50 to 70° C., to evaporate residual liquid from the extract. In some embodiments, the extract is dried, for example, by spray drying.

The microbe-based products may be formulated in a variety of ways, including liquid, solids, granular, dust, or slow release products by means that will be understood by those of skill in the art having the benefit of the instant disclosure.

Solid formulations may be milled, granulated or powdered, and/or may have different forms and shapes such as cylinders, rods, blocks, capsules, tablets, pills, pellets, strips, spikes, etc. The granulated or powdered material may be pressed into tablets or used to fill pre-manufactured gelatin capsules or shells. Semi solid formulations can be prepared in paste, wax, gel, or cream preparations.

The solid or semi-solid compositions of the invention can be coated using film-coating compounds such as polyethylene glycol, gelatin, sorbitol, gum, sugar or polyvinyl alcohol. This is particularly essential for tablets or capsules used in pesticide formulations. Film coating can protect the handler from coming in direct contact with the active ingredient in the formulations. In addition, a bittering agent such as denatonium benzoate or quassin may also be incorporated in the pesticidal formulations, the coating or both.

The adjuvant compositions can also be prepared in powder formulations and used as-is, or, optionally, filled into pre-manufactured gelatin capsules.

The health-promoting compound can be administered simultaneously with the adjuvant composition, for example, as part of a single mixture or formulation.

In one embodiment, the adjuvant composition and the health-promoting compound are formulated together into a capsule, nanocapsule or liposome. In some embodiments, the growth by-products of the yeast produced during cultivation have amphiphilic properties that facilitate the formation of liposomes. In one embodiment, additional biological polymers can be included to provide further structure for encapsulation.

This nanocapsule/liposome delivery system can enhance the bioavailability of a health-promoting compound by protecting the compound from components in the blood, such as proteins and other molecules, that otherwise might bind to the compound and prevent it from penetrating a target site. Additionally, the nanocapsule/liposome delivery system can allow for health-promoting compounds that might otherwise by degraded by acids or enzymes in the GI tract to be administered orally, as it creates a barrier against the acids or enzymes. Furthermore, in some embodiments, the nanocapsule/liposome delivery system formulation facilitates timed release of the health-promoting compound, thereby reducing the potential toxicity or potential negative side-effects of a compound in a subject.

In one embodiment, the adjuvant composition can be formulated to comprise an orally deliverable health-promoting compound and/or to be administered simultaneously with one as an orally consumable product. An orally deliverable health-promoting compound is any physiologically active substance delivered via initial absorption into the gastrointestinal tract, or into the mucus membranes of the mouth (e.g., by way of sublingual or buccal administration).

Orally consumable products according to the invention are any preparations or compositions suitable for consumption, for nutrition, for oral hygiene or for pleasure, and are products intended to be introduced into the human or animal oral cavity, to remain there for a certain period of time and then to either be swallowed (e.g., food ready for consumption or pills) or to be removed from the oral cavity again (e.g., chewing gums or products of oral hygiene or medical mouth washes). While an orally-deliverable health-promoting compound can be formulated into an orally consumable product, and an orally consumable product can comprise an orally-deliverable health-promoting compound, the two terms are not meant to be used interchangeably herein.

Orally consumable products include all substances or products intended to be ingested by humans or animals in a processed, semi-processed or unprocessed state. This also includes substances that are added to orally-consumable products (particularly food and pharmaceutical products) during their production, treatment or processing and intended to be introduced into the human or animal oral cavity.

Orally-consumable products can also include substances intended to be swallowed by humans or animals and then digested in an unmodified, prepared or processed state; the orally consumable products according to the invention therefore also include casings, coatings or other encapsulations that are intended also to be swallowed together with the product or for which swallowing is to be anticipated.

In one embodiment, the orally-consumable product is a capsule, pill, syrup, emulsion or liquid suspension containing a desired orally-deliverable substance. In one embodiment, the orally-consumable product can comprise an orally-deliverable substance in powder form, which can be mixed with water or another liquid to produce a drinkable orally-consumable product.

In some embodiments, the orally-consumable product according to the invention can comprise one or more formulations intended for nutrition or pleasure. These particularly include baking products (e.g., bread, dry biscuits, cake, and other pastries), sweets (e.g., chocolates, chocolate bar products, other bar products, fruit gum, coated tablets, hard caramels, toffees and caramels, and chewing gum), alcoholic or non-alcoholic beverages (e.g., cocoa, coffee, green tea, black tea, black or green tea beverages enriched with extracts of green or black tea, Rooibos tea, other herbal teas, fruit-containing lemonades, isotonic beverages, soft drinks, nectars, fruit and vegetable juices, and fruit or vegetable juice preparations), instant beverages (e.g., instant cocoa beverages, instant tea beverages, and instant coffee beverages), meat products (e.g., ham, fresh sausage preparations or raw sausage preparations, and seasoned oder, marinated fresh meat or salted meat products), eggs or egg products (e.g., dried whole egg, egg white, and egg yolk), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, whey, butter, buttermilk, and partly or wholly hydrolyzed products containing milk proteins), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sauces, and fruit fillings), vegetable preparations (e.g., ketchup, sauces, dried vegetables, deep-freeze vegetables, pre-cooked vegetables, and boiled vegetables), snack articles (e.g., baked or fried potato chips (crisps) or potato dough products, and extrudates on the basis of maize or peanuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening or whitening beverages or other food). The present compositions may also serve as semi-finished products for the production of other compositions intended for nutrition or pleasure.

In one embodiment, the adjuvant composition can be formulated to comprise a health-promoting compound and/or to be administered simultaneously with one via a route of administration, including, for example, injection (e.g., intravenous (IV), intramuscular (IM), intraperitoneal, intrathecal or subcutaneous), transdermal, rectal, urogenital (e.g., vaginal), ocular, aural, nasal, inhalation and cutaneous routes.

The subject composition can further comprise one or more pharmaceutically acceptable carriers and/or excipients, and can be formulated into preparations in, for example, solid, semi-solid, liquid or gaseous forms, such as tablets, capsules, powders, granules, ointments, gels, lotions, solutions, suppositories, drops, patches, injections, inhalants and aerosols.

The term “pharmaceutically acceptable” as used herein means compatible with the other ingredients of a pharmaceutical composition and not deleterious to the recipient thereof.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilisers (such as, e.g., Tween 80, Polysorbate 80), colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavorings, aromatisers, thickeners, coatings, preservatives (such as, e.g., Thimerosal, benzyl alcohol), antioxidants (such as, e.g., ascorbic acid, sodium metabisulfite), tonicity controlling agents, absorption delaying agents, adjuvants, bulking agents (such as, e.g., lactose, mannitol) and the like. The use of carriers and/or excipients in the field of drugs and supplements is well known. Except for any conventional media or agent that is incompatible with the target health-promoting compound or with the adjuvant composition, its use in the subject compositions may be contemplated.

In one embodiment, the adjuvant composition can be made into aerosol formulations so that, for example, it can be nebulized or inhaled. Suitable pharmaceutical formulations for administration in the form of aerosols or sprays are, for example, solutions, suspensions or emulsions. Formulations for oral or nasal aerosol or inhalation administration may also be formulated with illustrative carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI) or any other of the numerous nebulizer delivery devices available in the art. Additionally, mist tents or direct administration through endotracheal tubes may also be used.

In one embodiment, the adjuvant composition can be formulated for administration via injection, for example, as a solution or suspension. The solution or suspension can comprise suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

Other suitable additives, which may be contained in the formulations according to the invention, include substances that are customarily used for such preparations. Example of such additives include adjuvants, emulsifying agents, fillers, plasticizers, lubricants, glidants, colorants, pigments, bittering agents, buffering agents, solubility controlling agents, pH adjusting agents, preservatives, stabilizers and ultra-violet light resistant agents.

In one embodiment, the composition may further comprise buffering agents including organic and amino acids or their salts. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In a further embodiment, pH adjusting agents include potassium hydroxide, ammonium hydroxide, potassium carbonate or bicarbonate, hydrochloric acid, nitric acid, sulfuric acid or a mixture.

In one embodiment, additional components such as sodium bicarbonate or carbonate, sodium sulfate, sodium phosphate, sodium biphosphate, can be included in the formulation.

### Local Production of Microbe-Based Products

In certain embodiments of the subject invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The microbe growth facilities of the subject invention can be located at the location where the microbe-based product will be used (e.g., a pharmaceutical plant). For example, the microbe growth facility may be less than 300, 250, 200, 150, 100, 75, 50, 25, 15, 10, 5, 3, or 1 mile from the location of use.

Because the microbe-based product can be generated locally, without resort to the microorganism stabilization, preservation, storage and transportation processes of conventional microbial production, a much higher density of microorganisms can be generated, thereby requiring a smaller volume of the microbe-based product for use in the on-site application or which allows much higher density microbial applications where necessary to achieve the desired efficacy. This allows for a scaled-down bioreactor (e.g., smaller fermentation vessel, smaller supplies of starter material, nutrients and pH control agents), which makes the system efficient and can eliminate the need to stabilize cells or separate them from their culture medium. Local generation of the microbe-based product also facilitates the inclusion of the growth medium in the product, when desired. The medium can contain agents produced during the fermentation that are particularly well-suited for local use.

Locally-produced high density, robust cultures of microbes are more effective in the field than those that have remained in the supply chain for some time. The microbe-based products of the subject invention are particularly advantageous compared to traditional products wherein cells have been separated from metabolites present in the fermentation growth media. Reduced transportation times allow for the production and delivery of fresh batches of microbes and/or their metabolites at the time and volume as required by local demand.

The microbe growth facilities of the subject invention produce fresh, microbe-based compositions, comprising the microbes themselves, microbial metabolites, and/or other components of the medium in which the microbes are grown. If desired, the compositions can have a high density of vegetative cells or propagules, or a mixture of vegetative cells and propagules.

In one embodiment, the microbe growth facility is located on, or near, a site where the microbe-based products will be used, for example, within 300 miles, 200 miles, or even within 100 miles.

Advantageously, distributed microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

Furthermore, by producing a composition locally, the formulation and potency can be adjusted in real time to a specific location and the conditions present at the time of application. This provides advantages over compositions that are pre-made in a central location and have, for example, set ratios and formulations that may not be optimal for a given location.

The cultivation time for the individual vessels may be, for example, from 1 to 7 days or longer. The cultivation product can be harvested in any of a number of different ways.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

What is claimed:

1. A method for enhancing the bioavailability of a health-promoting compound, the method comprising: administering to the subject a therapeutically-effective amount of an adjuvant composition comprising yeast extract, and a therapeutically-effective amount of the health-promoting compound.

2. The method of claim 1, wherein the yeast extract comprises proteins, nucleic acids, amino acids, peptides, carbohydrates and/or metabolites produced by the yeast during cultivation thereof.

3. The method of claim 1, wherein the health-promoting compound and the adjuvant composition are administered to a subject through a route selected from oral, intravenous, rectal, vaginal, ocular, transdermal, aural, nasal, mucosal, inhalation and cutaneous.

4. The method of claim 1, used to enhance penetration of the health-promoting compound into epithelial cells in the subject's gastrointestinal tract.

5. The method of claim 1, used to enhance penetration of the health-promoting compound across the subject's blood-brain barrier.

6. The method of claim 1, used to reduce the amount of the health-promoting compound that is required for it to be therapeutically-effective in the subject.

7. The method of claim 1, wherein the yeast extract is produced by: a) cultivating Saccharomyces cerevisiae yeast cells in a nutrient medium comprising one or more oils comprising a saturated fatty acid as a carbon source to produce a desired cell density; b) inactivating the yeast cells to produce a hydrolysate; c) centrifuging the hydrolysate to produce a supernatant and a pellet, the pellet comprising yeast cell walls, and d) removing the pellet,
wherein the supernatant comprises the yeast extract.

8. The method of claim 7, wherein the one or more oils are vegetable oil, sunflower oil, soybean oil, peanut oil or coconut oil.

9. The method of claim 7, further comprising concentrating the yeast extract using a rotary evaporator operated at a temperature of 50° C. to 70° C.

10. The method of claim 7, further comprising drying the yeast extract.

11. The method of claim 1, wherein the adjuvant composition further comprises a pharmaceutically-acceptable carrier.

12. The method of claim 1, wherein the adjuvant composition and the health-promoting compound are mixed together as one formulation when administered to the subject.

13. The method of claim 12, wherein the adjuvant composition and the health-promoting compound are encapsulated together into a capsule, nanocapsule or liposome delivery system.

14. The method of claim 1, wherein the adjuvant composition is administered to the subject as a separate composition from the health-promoting compound.

15. The method of claim 1, wherein the health-promoting compound is a pain-reliever, antihistamine, antiviral, anticancer and/or chemotherapeutic compound, antibiotic, antimicrobial, antiseizure compound, anti-inflammatory compound, vaccine, statin, hormone, antidepressant, vitamin, mineral, nutrient or water.

16. The method of claim 1, wherein the health-promoting compound is cannabidiol (CBD).

17. A composition comprising an adjuvant and a health-promoting compound, said adjuvant comprising yeast extract of S. cerevisiae.

18. The composition of claim 17, wherein the health-promoting compound is a pain-reliever, antihistamine, antiviral, anticancer and/or chemotherapeutic compound, antibiotic, antimicrobial, antiseizure compound, anti-inflammatory compound, vaccine, statin, hormone, antidepressant, vitamin, mineral, nutrient or water.

19. The composition of claim 17, wherein the health-promoting compound is cannabidiol (CBD).
